(19) United States
(12) Patent Application Publication
(10) Pub. No.: US 2023/0105879 A1
(43) Pub. Date: Apr. 6, 2023

(54) Antiviral Composition

(71) Applicant: Luke Klele, Emerson, NJ (US)

(72) Inventor: Luke Klele, Emerson, NJ (US)

(21) Appl. No.: 18/078,202

(22) Filed: Dec. 9, 2022

Related U.S. Application Data
(63) Continuation of application No. PCT/US21/35040, filed on May 29, 2021.
(60) Provisional application No. 63/032,822, filed on Jun. 1, 2020.
(60) Provisional application No. 63/128,522, filed on Dec. 21, 2020.

(51) Int. Cl. A61K 33/30; A61K 9/00; A61K 33/34; A61K 31/375; A61K 36/282; A61K 31/05; A61K 36/35; A61K 36/537; A61K 31/59

(52) U.S. Cl. CPC A61K 33/30; A61K 9/0043; A61K 33/34; A61K 31/375; A61K 36/282; A61K 31/05; A61K 36/35; A61K 36/537; A61K 31/59

(57) ABSTRACT

Disclosed herein are antiviral compositions for treating CoV. In some embodiments, the antiviral compositions include at least 5 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

### CROSS REFERENCE TO RELATED APPLICATIONS

The present disclosure is a continuation of International Application No. PCT/US21/35040 filed on May 29, 2021, which application claims the benefit of the filing date of U.S. Patent Application No. 63/128,522 filed on Dec. 21, 2020; and also claims the benefit of the filing date of U.S. patent application Ser. No. 16/889,039 filed on Jun. 1, 2020, the disclosures of which are incorporated by reference herein in their entireties.

### FIELD OF THE DISCLOSURE

The invention relates generally to antiviral compositions and treatment of viral infections in patients by administering an antiviral composition to the patient in need of treatment thereof.

### BACKGROUND OF THE DISCLOSURE

Coronaviruses (CoV) are zoonotic viral pathogens that can be found in cats, dogs, bats, pigs, poultry and rodents, can be transmitted from animals to humans, can cause respiratory and gastrointestinal system infections, and rarely, with hepatic, neurological and nephrotic involvement. Coronaviruses are single-stranded, positive-polarity enveloped RNA viruses in the coronavirinae subfamily of the coronaviridae family in the nidovirales class. These viruses are named as “coronavirus,” based on the word “corona”, which means “crown” in Latin, due to the rod-shaped extensions on their surfaces.

Coronaviruses are enveloped positive strand RNA viruses and have dangerous effects on both humans and animals. Coronaviruses have caused infections such as severe sudden respiratory failure syndrome (SARS), Middle East respiratory syndrome (MERS) and 2019 coronavirus disease (COVID-19) that have resulted in global epidemics that have a significant impact on people's health and lives. In February 2020, the World Health Organization identified COVID-19 disease caused by the SARS-Cov-2 virus, which means 2019 corona virus disease. Coronaviruses cause severe respiratory failure and damage.

In humans, CoV causes respiratory infections, which are typically mild but can be lethal in rare forms such as SARS (severe acute respiratory syndrome)-CoV, MERS (Middle East Respiratory Syndrome)-CoV, and COVID-19. CoV are transmitted by aerosols of respiratory secretions, by the fecal-oral route, and by mechanical transmission. Most virus growth occurs in epithelial cells. Occasionally the liver, kidneys, heart or eyes may be infected, as well as other cell types such as macrophages. In cold-type respiratory infections, growth appears to be localized to the epithelium of the upper respiratory tract. Coronavirus infection is very common and occurs worldwide. The incidence of infection is strongly seasonal, with the greatest incidence in children in winter. Adult infections are less common. The number of coronavirus serotypes and the extent of antigenic variation is unknown. Re-infections appear to occur throughout life, implying multiple serotypes (at least four are known) and/or antigenic variation, hence the prospects for immunization against all serotypes with a single vaccine is highly unlikely. SARS is a type of viral pneumonia, with symptoms including fever, a dry cough, dyspnea (shortness of breath), headache, and hypoxaemia (low blood oxygen concentration). Typical laboratory findings include lymphopaenia (reduced lymphocyte numbers) and mildly elevated aminotransferase levels (indicating liver damage). Death may result from progressive respiratory failure due to alveolar damage. The typical clinical course of SARS involves an improvement in symptoms during the first week of infection, followed by a worsening during the second week. A substantial need remains for effective antiviral treatments (compositions and methods) against human CoV.

### BRIEF SUMMARY OF THE DISCLOSURE

In one aspect of the present disclosure is an antiviral composition comprising: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

In some embodiments, the at least one mineral is selected from zinc or copper. In some embodiments, the antiviral composition comprises a therapeutically effective amount of at least two minerals. In some embodiments, the at least two minerals are zinc and copper.

In some embodiments, the at least one vitamin is selected from Vitamin D or Vitamin C. In some embodiments, the antiviral composition comprises a therapeutically effective amount of at least two vitamins. In some embodiments, the at least two vitamins are Vitamin D and Vitamin C.

In some embodiments, the one or more Artemisinins are selected from the group consisting of dihydroartemisinin (DHA), artemether, artesunate, artemisone, arteether, and artelinic acid. In some embodiments, the antiviral composition further comprises a cannabidiol. In some embodiments, the antiviral composition further comprises a pharmaceutically acceptable carrier or excipient.

Another aspect of the present disclosure is a method of treating a CoV infection comprising administering an antiviral composition to a subject in need of treatment thereof, wherein the antiviral composition comprises a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of reducing a viral load comprising administering an antiviral composition to a subject in need of treatment thereof, wherein the antiviral composition comprises a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of reducing a symptom of a CoV infection comprising administering an antiviral composition to a subject in need of treatment thereof, wherein the antiviral composition comprises a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of preventing a CoV infection comprising administering an antiviral composition to a subject in need of treatment thereof, wherein the antiviral composition comprises a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of preventing an onset of a symptom of a CoV infection comprising administering an antiviral composition to a subject in need of treatment thereof, wherein the antiviral composition comprises a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of preventing hospitalization of a patient infected with CoV comprising administering an antiviral composition to a subject in need of treatment thereof, wherein the antiviral composition comprises a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is an antiviral composition consisting essentially of: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of treating a CoV infection comprising administering to a subject in need of treatment thereof, wherein the antiviral composition consists essentially of: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of reducing a viral load comprising administering an antiviral composition to a subject in need of treatment thereof, wherein the antiviral composition consists essentially of: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of reducing a symptom of a CoV infection comprising administering an antiviral composition to a subject in need of treatment thereof, wherein the antiviral composition consists essentially of: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of treating a CoV infection comprising administering an antiviral composition to a subject in need of treatment thereof, wherein the antiviral composition consists of: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of reducing a viral load comprising administering an antiviral composition to a subject in need of treatment thereof, wherein the antiviral composition consists of: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

Another aspect of the present disclosure is a method of reducing a symptom of a CoV infection comprising administering an antiviral composition to a subject in need of treatment thereof, wherein the antiviral composition consists of: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

An intranasal dosage form comprising (i) at least 4 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

In some embodiments, the at least one mineral is selected from zinc or copper. In some embodiments, the antiviral composition comprises a therapeutically effective amount of at least two minerals. In some embodiments, the at least two minerals are zinc and copper.

In some embodiments, the at least one vitamin is selected from Vitamin D or Vitamin C. In some embodiments, the antiviral composition comprises a therapeutically effective amount of at least two vitamins. In some embodiments, the at least two vitamins are Vitamin D and Vitamin C.

In some embodiments, the one or more Artemisinins are selected from the group consisting of dihydroartemisinin (DHA), artemether, artesunate, artemisone, arteether, and artelinic acid. In some embodiments, the antiviral composition further comprises a cannabidiol. In some embodiments, the antiviral composition further comprises a pharmaceutically acceptable carrier or excipient.

Another aspect of the present disclosure is a method of treating a CoV infection comprising administering an intranasal composition to a subject in need of treatment thereof, wherein the intranasal composition comprises (i) at least 4 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

Another aspect of the present disclosure is a method of reducing a viral load comprising administering an intranasal composition to a subject in need of treatment thereof, wherein the intranasal composition comprises (i) at least 4 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

Another aspect of the present disclosure is a method of reducing a symptom of a CoV infection comprising administering an intranasal composition to a subject in need of treatment thereof, wherein the intranasal composition comprises (i) at least 4 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

Another aspect of the present disclosure is a method of preventing a CoV infection comprising administering an intranasal composition to a subject in need of treatment thereof, wherein the intranasal composition comprises (i) at least 4 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

Another aspect of the present disclosure is a method of preventing hospitalization of a patient infected with CoV comprising administering an intranasal composition to a subject in need of treatment thereof, wherein the intranasal composition comprises (i) at least 4 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

An intranasal dosage form comprising (i) at least 5 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

In some embodiments, the at least one mineral is selected from zinc or copper. In some embodiments, the antiviral composition comprises a therapeutically effective amount of at least two minerals. In some embodiments, the at least two minerals are zinc and copper.

In some embodiments, the at least one vitamin is selected from Vitamin D or Vitamin C. In some embodiments, the antiviral composition comprises a therapeutically effective amount of at least two vitamins. In some embodiments, the at least two vitamins are Vitamin D and Vitamin C.

In some embodiments, the one or more Artemisinins are selected from the group consisting of dihydroartemisinin (DHA), artemether, artesunate, artemisone, arteether, and artelinic acid. In some embodiments, the antiviral composition further comprises a cannabidiol. In some embodiments, the antiviral composition further comprises a pharmaceutically acceptable carrier or excipient.

An intranasal dosage form comprising (i) at least 6 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

In some embodiments, the at least one mineral is selected from zinc or copper. In some embodiments, the antiviral composition comprises a therapeutically effective amount of at least two minerals. In some embodiments, the at least two minerals are zinc and copper.

In some embodiments, the at least one vitamin is selected from Vitamin D or Vitamin C. In some embodiments, the antiviral composition comprises a therapeutically effective amount of at least two vitamins. In some embodiments, the at least two vitamins are Vitamin D and Vitamin C.

In some embodiments, the one or more Artemisinins are selected from the group consisting of dihydroartemisinin (DHA), artemether, artesunate, artemisone, arteether, and artelinic acid. In some embodiments, the antiviral composition further comprises a cannabidiol. In some embodiments, the antiviral composition further comprises a pharmaceutically acceptable carrier or excipient.

An intranasal dosage form consisting essentially of (i) at least 4 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

In some embodiments, the at least one mineral is selected from zinc or copper. In some embodiments, the antiviral composition comprises a therapeutically effective amount of at least two minerals. In some embodiments, the at least two minerals are zinc and copper.

In some embodiments, the at least one vitamin is selected from Vitamin D or Vitamin C. In some embodiments, the antiviral composition comprises a therapeutically effective amount of at least two vitamins. In some embodiments, the at least two vitamins are Vitamin D and Vitamin C.

In some embodiments, the one or more Artemisinins are selected from the group consisting of dihydroartemisinin (DHA), artemether, artesunate, artemisone, arteether, and artelinic acid. In some embodiments, the antiviral composition further comprises a cannabidiol. In some embodiments, the antiviral composition further comprises a pharmaceutically acceptable carrier or excipient.

An intranasal dosage form consisting essentially of (i) at least 5 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

In some embodiments, the at least one mineral is selected from zinc or copper. In some embodiments, the antiviral composition comprises a therapeutically effective amount of at least two minerals. In some embodiments, the at least two minerals are zinc and copper.

In some embodiments, the at least one vitamin is selected from Vitamin D or Vitamin C. In some embodiments, the antiviral composition comprises a therapeutically effective amount of at least two vitamins. In some embodiments, the at least two vitamins are Vitamin D and Vitamin C.

In some embodiments, the one or more Artemisinins are selected from the group consisting of dihydroartemisinin (DHA), artemether, artesunate, artemisone, arteether, and artelinic acid. In some embodiments, the antiviral composition further comprises a cannabidiol. In some embodiments, the antiviral composition further comprises a pharmaceutically acceptable carrier or excipient.

An intranasal dosage form consisting essentially of (i) at least 6 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.

In some embodiments, the at least one mineral is selected from zinc or copper. In some embodiments, the antiviral composition comprises a therapeutically effective amount of at least two minerals. In some embodiments, the at least two minerals are zinc and copper.

In some embodiments, the at least one vitamin is selected from Vitamin D or Vitamin C. In some embodiments, the antiviral composition comprises a therapeutically effective amount of at least two vitamins. In some embodiments, the at least two vitamins are Vitamin D and Vitamin C.

In some embodiments, the one or more Artemisinins are selected from the group consisting of dihydroartemisinin (DHA), artemether, artesunate, artemisone, arteether, and artelinic acid. In some embodiments, the antiviral composition further comprises a cannabidiol. In some embodiments, the antiviral composition further comprises a pharmaceutically acceptable carrier or excipient.

### DETAILED DESCRIPTION

### Definitions

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “administering” means providing a composition, formulation, or specific agent to a subject in need of treatment, including those described herein. Administration may, for example, be by way of absorption through oral or other (e.g., rectal or vaginal) mucosa (referred to herein as “transmucosal” delivery or administration. Alternatively, administration may be by intramuscular, intravenous or intraperitoneal delivery means, which are collectively referred to herein as “parenteral” administration or delivery. Topical administration, for example for the treatment of shingles, or other viral infection affecting the skin, is not excluded from the meaning of “administering” presented herein.

As used herein, the term “antiviral activity” refers to the ability of a composition or treatment regimen to ameliorate the symptoms of a viral infection. Antiviral activity includes but is not limited to an activity resulting in a reduction by at least 10% in viral titer or a reduction by at least 10% in the severity or duration of the symptoms of a viral infection. The symptoms of a viral infection include not only those directly caused by viral replication and accompanying cell death, but also secondary symptoms, such as those caused by opportunistic bacterial infections that occur subsequent to the death of infected cells.

As used herein, the term “extract” refers to an alcoholic extract, an alcohol/water extract or an oil-based extract of a material, in particular, a plant component.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the expression vectors of the present disclosure, its use in therapeutic compositions is contemplated.

As used herein, the term “subject” refers to a mammal such as a human, mouse or primate. Typically, the mammal is a human (Homo sapiens). A human subject may be an adult patient or a pediatric patient.

As used herein, the terms “therapeutically effective dose” or “dose amount” refer to an amount of a composition, or a component of the composition, which is effective to achieve an improvement in a subject or his physiological systems including, but not limited to, improved improvement or elimination of symptoms, delayed onset of a disorder, slower progress of symptoms and other indicators selected as appropriate by those skilled in the art. In the context of a viral infection, e.g., an infection caused by Coy, a therapeutically effective dose can be administered according to any dosing regimen typically used in the treatment of a viral infection. A therapeutically effective dose can be administered once, twice, thrice or more daily. It can be administered every other day, every third day, every fourth day, every fifth day, semiweekly, weekly, biweekly, every three weeks, every four weeks, monthly, bimonthly, semimonthly, every three months, every four months, semiannually, annually, or according to a combination of any of the above to arrive at a suitable dosing schedule. For example, a therapeutically effective dose can be administered one or more times daily (up to 10 times daily for the highest dose) for one or more weeks.

A subject treated with any of the compositions of formulations of the present disclosure will exhibit a therapeutic response. By “therapeutic response” it is meant that a subject suffering from a viral infection will enjoy at least one of the following clinical benefits as a result of treatment: reduction of the active viral titer in the subject's blood or plasma, eradication of active virus from the subject's blood or plasma, amelioration of the infection, reduction in the occurrence of symptoms associated with the infection, partial or full remission of the infection or increased time to progression of the infection, and/or reduction in the infectivity of the virus causing said viral infection. The therapeutic response can be a full or partial therapeutic response.

As used herein, the terms “treatment,” “treating,” or “treat,” with respect to a specific condition, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition. The term “treatment” is used in some embodiments to refer to administration of a compound of the present disclosure to mitigate a disease or a disorder in a host, preferably in a mammalian subject, more preferably in humans. Thus, the term “treatment” can include includes: preventing a disorder from occurring in a host, particularly when the host is predisposed to acquiring the disease but has not yet been diagnosed with the disease; inhibiting the disorder; and/or alleviating or reversing the disorder. Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure can occur prior to onset of a disease. The term does not mean that the disease state must be completely avoided.

In general, the present disclosure provides for antiviral compositions comprising at least two active components, where the active components are selected from: spikenard or an extract thereof; one or more minerals (e.g., zinc, copper); one or more vitamins (e.g., vitamin D, vitamin C); oregano oil or at least one active component included within oregano oil (e.g. carvacrol, thymol, terpinene); one or more Artemisinins or derivatives or analogs thereof; one or more Salvia species or an extract thereof (e.g., salvia scalarea); cardamom oil or an extract thereof; and a cannabidiol. In some embodiments, the antiviral composition includes at least three of the active components. In some embodiments, the antiviral composition includes at least four of the active components. In some embodiments, the antiviral composition includes at least five of the active components. In some embodiments, the antiviral composition includes at least six of the active components. In some embodiments, the antiviral composition includes at least seven of the active components. In some embodiments, the antiviral composition includes at least eight of the active components.

In some embodiments, the present disclosure provides for antiviral compositions consisting essentially of at least two active components, where the active components are selected from: spikenard or an extract thereof; one or more minerals (e.g., zinc, copper); one or more vitamins (e.g., vitamin D, vitamin C); oregano oil or at least one active component included within oregano oil (e.g. carvacrol, thymol, terpinene) (collectively referred to herein as “oregano oil”); an Artemisinin or a derivative or analog thereof; a Salvia species or an extract thereof (e.g., salvia scalarea), cardamom oil or an extract thereof, and a cannabidiol. In some embodiments, the antiviral composition consists essentially of at least three of the active components. In some embodiments, the antiviral composition consists essentially of at least four of the active components. In some embodiments, the antiviral composition consists essentially of at least five of the active components. In some embodiments, the antiviral composition consists essentially of at least six of the active components. In some embodiments, the antiviral composition consists essentially of at least seven of the active components. In some embodiments, the antiviral composition consists essentially of at least eight of the active components. In some embodiments, the antiviral composition consists essentially of at least eight of the active components.

In some embodiments, the present disclosure provides for antiviral compositions consisting of at least two active components, where the active components are selected from: spikenard or an extract thereof; one or more minerals (e.g., zinc, copper); one or more vitamins (e.g., vitamin D, vitamin C); oregano oil or at least one active component included within oregano oil (e.g. carvacrol, thymol, terpinene); an Artemisinin or a derivative or analog thereof, a Salvia species or an extract thereof (e.g., salvia scalarea), cardamom oil or an extract thereof, and a cannabidiol. In some embodiments, the antiviral composition consists essentially of at least three of the active components. In some embodiments, the antiviral composition consists of at least four of the active components. In some embodiments, the antiviral composition consists of at least five of the active components. In some embodiments, the antiviral composition consists of at least six of the active components. In some embodiments, the antiviral composition consists of at least seven of the active components. In some embodiments, the antiviral composition consists of at least eight of the active components.

Oregano oil is a volatile oil extracted from the oregano genus Oregano. Oregano oil includes one or more of the compounds carvacrol, thymol, and/or terpinene. Oregano oil is a volatile oil obtained by steam distillation of oregano. Origanol is an aromatic phenolic substance in oregano oil, which mainly contains phenolic components such as thymol and carvacrol. Thymol and parsley are used alone or mixed in different proportions, all are believed to have good antibacterial, anti-oxidation and anti-tumor effects.

In some embodiments, an amount of oregano oil within any antiviral composition ranges from between about 1% to about 20% by total weight of the antiviral composition. In other embodiments, an amount of oregano oil within any antiviral composition ranges from between about 2% to about 18% by total weight of the antiviral composition. In yet other embodiments, an amount of oregano oil within any antiviral composition ranges from between about 4% to about 16% by total weight of the antiviral composition. In yet other embodiments, an amount of oregano oil within any antiviral composition ranges from between about 6% to about 12% by total weight of the antiviral composition. In yet other embodiments, an amount of oregano oil within any antiviral composition is about 2%. In yet other embodiments, an amount of oregano oil within any antiviral composition is about 4%. In yet other embodiments, an amount of oregano oil within any antiviral composition is about 6%. In yet other embodiments, an amount of oregano oil within any antiviral composition is about 8%. In yet other embodiments, an amount of oregano oil within any antiviral composition is about 10%. In yet other embodiments, an amount of oregano oil within any antiviral composition is about 12%. In yet other embodiments, an amount of oregano oil within any antiviral composition is about 14%. In yet other embodiments, an amount of oregano oil within any antiviral composition is about 16%. In yet other embodiments, an amount of oregano oil within any antiviral composition is about 18%. In yet other embodiments, an amount of oregano oil within any antiviral composition is about 20%.

Artemisinins are derived from extracts of sweet wormwood (Artemisia annua). Artemisinins include dihydroartemisinin (DHA), artemether, artesunate, artemisone, arteether, and artelinic acid.

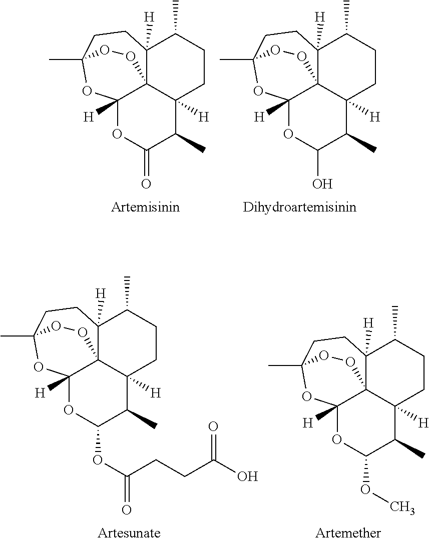

In some embodiments, an amount of one or more Artemisinins within any antiviral composition ranges from between about 1% to about 20% by total weight of the antiviral composition. In other embodiments, an amount of one or more Artemisinins within any antiviral composition ranges from between about 2% to about 18% by total weight of the antiviral composition. In yet other embodiments, an amount of one or more Artemisinins within any antiviral composition ranges from between about 4% to about 16% by total weight of the antiviral composition. In yet other embodiments, an amount of one or more Artemisinins within any antiviral composition ranges from between about 6% to about 12% by total weight of the antiviral composition. In yet other embodiments, an amount of one or more Artemisinins within any antiviral composition is about 2%. In yet other embodiments, an amount of one or more Artemisinins within any antiviral composition is about 4%. In yet other embodiments, an amount of one or more Artemisinins within any antiviral composition is about 6%. In yet other embodiments, an amount of one or more Artemisinins within any antiviral composition is about 8%. In yet other embodiments, an amount of one or more Artemisinins within any antiviral composition is about 10%. In yet other embodiments, an amount of one or more Artemisinins within any antiviral composition is about 12%. In yet other embodiments, an amount of one or more Artemisinins within any antiviral composition is about 14%. In yet other embodiments, an amount of one or more Artemisinins within any antiviral composition is about 16%. In yet other embodiments, an amount of one or more Artemisinins within any antiviral composition is about 18%. In yet other embodiments, an amount of one or more Artemisinins within any antiviral composition is about 20%.

Among spices, cardamom (Elettaria cardamomum), perennial herbaceous plant of order Zingiberaceae, also known as green cardamom, is used for prevention and control of several infections. It is commonly grown in tropical regions over the globe such as South-Western parts of the Indian Peninsula, Sri Lanka, Thailand, Vietnam, Mexico and Tanzania. In addition to provide distinctive flavor and fragrance in various foods, it is used as folk medicine, food preservative, and flavor modifier. In the fourth century, cardamom was used as a medicinal herb in India and was an item of Roman and Greek trade. It was mostly utilized as an aromatic stimulant, carminative, astringent, diuretic and anti-inflammatory in South Asia. It has also been used for the treatment of asthma, cardiac disorders, indigestion (stomatitis), rectal diseases and congestive jaundice as well as an insect controlling agent (Jamal et al. 2005; Verma et al. 2009; Sharma et al. 2011; Savan and Kucukbay 2013).

Cardamom fruit pods contained tiny, brown aromatic seeds that give pungent and sweet taste. It is commonly used as breath freshener to maintain oral health as antimicrobial agent that provides remedy for dental caries (Aneja and Radhika 2009). Furthermore, various clinical trials have confirmed that cardamom exhibits hypocholesterolemic, hypoglycemic, anti-inflammatory and antimutagenic properties against highly prevalent diseases (Hossain et al. 2008; Amma et al. 2010; Sharma et al. 2011; El-Yamani 2011).

Owing to its antioxidant properties, cardamom can remarkably increase levels of glutathione and antioxidant enzymes in the body by stimulating glutathione transferase and glutathione peroxidase activity (Amma et al. 2010). Ultimately, antioxidants interfere with free radicals production and inactivate them, thus protecting the biological systems against deleterious effects of oxidative processes. These substances are mostly natural products like ascorbates, carotenoids, tocopherols, polyphenols that subsidize the prevention and cure of diseases because of good antioxidant activity (Khalaf et al. 2008; Hossain et al. 2008).

In some embodiments, an amount of cardamom oil within any antiviral composition ranges from between about 5% to about 35% by total weight of the antiviral composition. In other embodiments, an amount of cardamom oil within any antiviral composition ranges from between about 10% to about 30% by total weight of the antiviral composition. In yet other embodiments, an amount of cardamom oil within any antiviral composition ranges from between about 15% to about 25% by total weight of the antiviral composition. In yet other embodiments, an amount of cardamom oil within any antiviral composition is about 10%. In yet other embodiments, an amount of cardamom oil within any antiviral composition is about 15%. In yet other embodiments, an amount of cardamom oil within any antiviral composition is about 20%. In yet other embodiments, an amount of cardamom oil within any antiviral composition is about 25%. In yet other embodiments, an amount of cardamom oil within any antiviral composition is about 30%. In yet other embodiments, an amount of cardamom oil within any antiviral composition is about 35%.

Clary Sage Oil (Salvia sclera) is an essential oil with astringent and anti-wrinkle properties. Clary sage a native to the Mediterranean region, has been found growing in the wild, and has been used extensively in traditional medicines. Dried leaves of clary sage have been used mostly as a tea ingredient; occasionally in tablets, capsules and tincture. Traditionally, sage is used as a tonic, digestive, antiseptic, astringent, and antispasmodic. It has been used to reduce perspiration (e.g., night sweats), to re-stop the flow of milk, to treat nervous conditions (e.g., trembling, depression, and vertigo), dsymenorrhea, diarrhea, gastritis, sore throat, and insect bites usually in the form of a tea or infusion. Sage can serve as source of natural antioxidants. Clary sage oleoresin is also widely used in baked goods meats and meat products, and condiments and relishes. Sage oil has been extensively used in most categories of food products including alcoholic (e.g., vermouths and bitters) and nonalcoholic beverages, frozen dairy desserts, candy, baked goods, gelatins and puddings, meat and meat products, and condiments and relishes.

Clary sage occurs as an annual (rare), biennial (fairly common) or perennial (fairly common) open-pollinated herbaceous plant that has typical quadrangular stems, opposite leaves and verticillastrate inflorescence.

Clary sage is cultivated mainly for the production of essential oil, sclareol and sclareol derivatives. U.S. Pat. No. 3,060,172 describes a process for the isolation of sclareol from clary sage; U.S. Pat. Nos. 5,525,728 and 5,247,100 describe processes for the production of the sclareolide from sclareol. The discloses of the ′172, ′728, and ′100 Patents are incorporated by reference herein in their entireties.

A variety of other Salvia species including Salvia miltiorrhiza Bunge (Dan-Shen), Salvia przewalskii and Salvia yunnanensis have also been used for medicinal purposes (Chemical constituents in the roots of Salvia przewalskii Maxim, YaO-Xue-Bao, 38 (5): 354-357, 2003; Medicinal resources of Salvia yunnanensis, Zhong-Yao-Cai, 25(9): 628-629, 2002). Particularly, Dan-Shen has been used for centuries in traditional Chinese medicine for the treatment of coronary heart disease, particularly angina pectoris and myocardial infarction (Ji et al., Salvia Miltiorrhiza and Ischemic Diseases, Acta Pharmacol Sin 12: 1089-1094, December 2000).

In some embodiments, an amount of one or more Salvia species within any antiviral composition ranges from between about 1% to about 20% by total weight of the antiviral composition. In other embodiments, an amount of one or more Salvia species within any antiviral composition ranges from between about 2% to about 18% by total weight of the antiviral composition. In yet other embodiments, an amount of one or more Salvia species within any antiviral composition ranges from between about 4% to about 16% by total weight of the antiviral composition. In yet other embodiments, an amount of one or more Salvia species within any antiviral composition ranges from between about 6% to about 12% by total weight of the antiviral composition. In yet other embodiments, an amount of one or more Salvia species within any antiviral composition is about 2%. In yet other embodiments, an amount of one or more Salvia species within any antiviral composition is about 4%. In yet other embodiments, an amount of one or more Salvia species within any antiviral composition is about 6%. In yet other embodiments, an amount of one or more Salvia species within any antiviral composition is about 8%. In yet other embodiments, an amount of one or more Salvia species within any antiviral composition is about 10%. In yet other embodiments, an amount of one or more Salvia species within any antiviral composition is about 12%. In yet other embodiments, an amount of one or more Salvia species within any antiviral composition is about 14%. In yet other embodiments, an amount of one or more Salvia species within any antiviral composition is about 16%. In yet other embodiments, an amount of one or more Salvia species within any antiviral composition is about 18%. In yet other embodiments, an amount of one or more Salvia species within any antiviral composition is about 20%.

Spikenard (Nardostachys jatamanse) is a class of aromatic, amber-colored essential oil. Without wishing to be bound by any particular theory, spikenard is considered a calming herb in ayurveda and unani because of its medicinal values. In ayurveda, it is often used against stress, spasm, epilepsy, convulsion and hysteria.

In some embodiments, an amount of Spikenard within any antiviral composition ranges from between about 1% to about 20% by total weight of the antiviral composition. In other embodiments, an amount of Spikenard within any antiviral composition ranges from between about 2% to about 18% by total weight of the antiviral composition. In yet other embodiments, an amount Spikenard within any antiviral composition ranges from between about 4% to about 16% by total weight of the antiviral composition. In yet other embodiments, an amount of Spikenard within any antiviral composition ranges from between about 6% to about 12% by total weight of the antiviral composition. In yet other embodiments, an amount of Spikenard within any antiviral composition is about 2%. In yet other embodiments, an amount of Spikenard within any antiviral composition is about 4%. In yet other embodiments, an amount of Spikenard within any antiviral composition is about 6%. In yet other embodiments, an amount of Spikenard within any antiviral composition is about 8%. In yet other embodiments, an amount of Spikenard within any antiviral composition is about 10%. In yet other embodiments, an amount of Spikenard within any antiviral composition is about 12%. In yet other embodiments, an amount of Spikenard within any antiviral composition is about 14%. In yet other embodiments, an amount of Spikenard within any antiviral composition is about 16%. In yet other embodiments, an amount of Spikenard within any antiviral composition is about 18%. In yet other embodiments, an amount of Spikenard within any antiviral composition is about 20%.

Without wishing to be bound by any particular theory, it is believed that cannabidiol (“CBD”) is non-psychoactive and may act as an anti-inflammatory agent. CBD is a compound belonging to a broader class of cannabinoids. Cannabinoids are a heteromorphic group of chemicals which activate the body's cannabinoid receptors. Initially cannabinoids were discovered in Cannabis sativa, the cannabis plant. There are three main types of cannabinoids: herbal cannabinoids that occur uniquely in the cannabis plant, synthetic cannabinoids that are manufactured and endogenous cannabinoids that are produced in vivo. Herbal cannabinoids are nearly insoluble in water but soluble in lipids, alcohol and non-polar organic solvents. These natural cannabinoids are concentrated in a viscous resin that is produced in glandular structures known as trichomes. In addition to cannabinoids, the resin is rich in terpenes, which are largely responsible for the odor of the cannabis plant.

Unlike cannabinoids (e.g. THC), cannabidiols do not bind either the brain receptor CB1 or the peripheral receptors CB2 and therefore does not cause the central or peripheral effects mediated by these receptors. Furthermore, CBD has no psychotropic (cannabimimetic) activity and its molecular structure and properties are substantially different from those of cannabinoids [Science 169: 611-612 (1970); “Marijuana/cannabinoids: neurobiology and neurophysiology,” ed. L. Murphy and A. Bartke, CRC Press, Boca Raton, 1-33 (1992)].

In addition to its immunomodulating and anti-inflammatory properties, CBD has been reported to exhibit anticonvulsive, anti-anxiety, and antipsychotic activity, and function as an efficient neuroprotective antioxidant. The in vitro suppressive effect of CBD on down-modulating the release of tumor necrosis factor α (TNFα), interleukin 1 (IL-1), and interferon γ (IFN)-γ from peripheral blood cells has also been reported. CBD has demonstrated activity in ameliorating collagen-induced arthritis in mice and has been shown to suppress T-cell responses and the production of TNFα and IFNγ. CBD also inhibits uptake of THC and anandamide and its hydrolysis. For example, U.S. Pat. No. 6,410,588 describes the use of cannabidiol for treating inflammatory diseases such as rheumatoid arthritis, multiple sclerosis and Crohn's Disease, and medicinal preparations containing CBD for use in treating such diseases. By means of another example, PCT/IL01/00537 describes pharmaceutical compositions comprising cannabidiol derivatives which have analgesic, antianxiety, anticonvulsive, neuroprotective, antipsychotic and anticancer activity.

In some embodiments, cannabidiol refers to 2-[3-methyl-6-(1-methylethenyl)-2-cyclohexen -1-yl]-5-pentyl-1,3-benzenediol as well as to pharmaceutically acceptable salts, solvates, metabolites (e.g., cutaneous metabolites), and metabolic precursors of 2-[3-methyl-6-(1-methylethenyl)-2-cyclohexen-1-yl]-5-pentyl-1,3-benzenediol. The synthesis of 2-[3-methyl-6-(1-methylethenyl)-2-cyclohexen-1-yl]-5-pentyl-1,3-benzenediol is described, for example, in Petilka et al., Helv. Chim. Acta, 52:1102 (1969) and in Mechoulam et al., J. Am. Chem. Soc., 87:3273 (1965), which are hereby incorporated by reference.

In some embodiments, an amount of cannabidiol within any antiviral composition ranges from between about 5% to about 35% by total weight of the antiviral composition. In other embodiments, an amount of cannabidiol within any antiviral composition ranges from between about 10% to about 30% by total weight of the antiviral composition. In yet other embodiments, an amount of cannabidiol within any antiviral composition ranges from between about 15% to about 25% by total weight of the antiviral composition. In yet other embodiments, an amount of cannabidiol within any antiviral composition is about 10%. In yet other embodiments, an amount of cannabidiol within any antiviral composition is about 15%. In yet other embodiments, an amount of cannabidiol within any antiviral composition is about 20%. In yet other embodiments, an amount of cannabidiol within any antiviral composition is about 25%. In yet other embodiments, an amount of cannabidiol within any antiviral composition is about 30%. In yet other embodiments, an amount of cannabidiol within any antiviral composition is about 35%.

Zinc is also known as zinc ions, is often supplied in the form of zinc acetate, zinc citrate, zinc gluconate, zinc glycerinate, zinc picolinate, or zinc sulfate, and is also contained in other zinc compounds. Effective daily antiviral dosages of Zinc for adults lie in the range of 80-200 mg/day.

In some embodiments, an amount of one or more minerals within any antiviral composition ranges from between about 1% to about 20% by total weight of the antiviral composition. In other embodiments, an amount of one or more minerals within any antiviral composition ranges from between about 2% to about 18% by total weight of the antiviral composition. In yet other embodiments, an amount of one or more minerals within any antiviral composition ranges from between about 4% to about 16% by total weight of the antiviral composition. In yet other embodiments, an amount of one or more minerals within any antiviral composition ranges from between about 6% to about 12% by total weight of the antiviral composition. In yet other embodiments, an amount of one or more minerals within any antiviral composition is about 2%. In yet other embodiments, an amount of one or more minerals within any antiviral composition is about 4%. In yet other embodiments, an amount of one or more minerals within any antiviral composition is about 6%. In yet other embodiments, an amount of one or more minerals within any antiviral composition is about 8%. In yet other embodiments, an amount of one or more minerals within any antiviral composition is about 10%. In yet other embodiments, an amount of one or more minerals within any antiviral composition is about 12%. In yet other embodiments, an amount of one or more minerals within any antiviral composition is about 14%. In yet other embodiments, an amount of one or more minerals within any antiviral composition is about 16%. In yet other embodiments, an amount of one or more minerals within any antiviral composition is about 18%. In yet other embodiments, an amount of one or more minerals within any antiviral composition is about 20%.

In some embodiments, a single mineral is included, e.g., zinc or copper. In other embodiments, both zinc and copper are included.

In some embodiments, an amount of one or more vitamins within any antiviral composition ranges from between about 1% to about 20% by total weight of the antiviral composition. In other embodiments, an amount of one or more vitamins within any antiviral composition ranges from between about 2% to about 18% by total weight of the antiviral composition. In yet other embodiments, an amount of one or more vitamins within any antiviral composition ranges from between about 4% to about 16% by total weight of the antiviral composition. In yet other embodiments, an amount of one or more vitamins within any antiviral composition ranges from between about 6% to about 12% by total weight of the antiviral composition. In yet other embodiments, an amount of one or more vitamins within any antiviral composition is about 2%. In yet other embodiments, an amount of one or more minerals within any antiviral composition is about 4%. In yet other embodiments, an amount of one or more vitamins within any antiviral composition is about 6%. In yet other embodiments, an amount of one or more vitamins within any antiviral composition is about 8%. In yet other embodiments, an amount of one or more vitamins within any antiviral composition is about 10%. In yet other embodiments, an amount of one or more vitamins within any antiviral composition is about 12%. In yet other embodiments, an amount of one or more vitamins within any antiviral composition is about 14%. In yet other embodiments, an amount of one or more vitamins within any antiviral composition is about 16%. In yet other embodiments, an amount of one or more vitamins within any antiviral composition is about 18%. In yet other embodiments, an amount of one or more vitamins within any antiviral composition is about 20%.

In some embodiments, a single mineral is included, e.g., Vitamin D or Vitamin C. In other embodiments, both Vitamin D and Vitamin C are included.

In some embodiments, the antiviral compositions may include an aluminum salt.

Pharmaceutically Acceptable Excipients, Carriers, and Additives

The antiviral compositions of the present disclosure may further comprise one or more pharmaceutically acceptable excipients including, but not limited to, diluents, binders, lubricants, disintegrants, flavoring agents, taste-masking agents, coloring agents, pH modifiers, stabilizers, absorption enhancers, viscosity modifiers, film forming polymers, bulking agents, surfactants, glidants, plasticizers, preservatives, essential oils and sweeteners. In some embodiments, the pharmaceutically acceptable excipients, carriers, and/or additives may be a food composition or a food product into which the antiviral compositions described herein may be introduced.

A person skilled in the art will be able to select the suitable excipients or mixtures of excipients for the desired antiviral composition. In general, the amount of any pharmaceutically acceptable excipient, carrier, and/or additive included within any antiviral composition may vary depending on the desired effect, route of administration, form of the final composition. In general, however, a total amount of pharmaceutically acceptable excipients, carriers, and/or additives formulated with the antiviral compositions may range from about 1% to about 99% by total weight of the composition. In other embodiments, the total amount of pharmaceutically acceptable excipients, carriers, and/or additives formulated with the antiviral compositions may range from about 1% to about9% by total weight of the composition. In other embodiments, the total amount of pharmaceutically acceptable excipients, carriers, and/or additives formulated with the antiviral compositions may range from about 1% to about 80% by total weight of the composition. In yet other embodiments, the total amount of pharmaceutically acceptable excipients, carriers, and/or additives within the antiviral compositions may range from about 1% to about 50% by total weight of the composition. In other embodiments, the total amount of pharmaceutically acceptable excipients, carriers, and/or additives formulated with the antiviral compositions may range from about 5% to about 50% by total weight of the composition. By way of example only, a formulation may comprise a 50:50 mixture of any of a antiviral composition and a pharmaceutically acceptable excipient, carrier, and/or additive.

In some embodiments, a ratio of an amount of an antiviral composition and an amount of a pharmaceutically acceptable excipient or carrier ranges from about 100:1 to about 1:100. In some embodiments, a ratio of an amount of an antiviral composition and an amount of a pharmaceutically acceptable excipient or carrier ranges from about 50:1 to about 1:50. In some embodiments, a ratio of an amount of an antiviral composition and an amount of a pharmaceutically acceptable excipient or carrier ranges from about 25:1 to about 1:25. In some embodiments, a ratio of an amount of an antiviral composition and an amount of a pharmaceutically acceptable excipient or carrier ranges from about 10:1 to about 1:10. In some embodiments, a ratio of an amount of an antiviral composition and an amount of a pharmaceutically acceptable excipient or carrier ranges from about 5:1 to about 1:5.

In some embodiments, the carrier is water. In some embodiments, an amount of water present in the composition ranges from about 90% to about 98% by total weight of the composition, from about 90% to about 97% by total weight of the composition, from about 90% to about 96% by total weight of the composition, from about 90% to about 95% by total weight of the composition, from about 90% to about 94% by total weight of the composition, from about 90% to about 93% by total weight of the composition, from about 90% to about 92% by total weight of the composition, and from about 90% to about 91% by total weight of the composition.

A diluent may be selected from, for example, calcium carbonate, calcium phosphate dibasic, calcium phosphate tribasic, calcium sulfate, microcrystalline cellulose, microcrystalline silicified cellulose, powdered cellulose, dextrate, dextrose, fructose, lactitol, lactose anhydrous, lactose monohydrate, lactose dihydrate, lactose trihydrate, mannitol, sorbitol, starch, pregelatinized starch, sucrose, talc, xylitol, maltose, maltodextrin, maltitol.

A binder may be selected from, for example, acacia, alginic acid, carbomer, carboxymethylcellulose calcium, carbomethylcellulose sodium, microcrystalline cellulose, powdered cellulose, ethyl cellulose, gelatin liquid glucose, guar gum, hydroxyethyl cellulose, hydroxypropyl cellulose, hydroxypropylmethyl cellulose, maltodextrin, methylcellulose, polydextrose, polyethtylene oxide, povidone, sodium alginate, starch paste, pregelatinized starch, sucrose, tragacanth, low-substituted hydroxypropyl cellulose, glucose, sorbitol.

A suitable filler may be selected from, for example, starch derivatives, such as corn starch, potato starch or rice starch, polysaccharides such as dextrins, maltodextrins, dextrates, microcrystalline cellulose, powdered cellulose, mixture of microcrystalline cellulose and guar gum, coprocessed blends of microcrystalline cellulose; and polyhydric alcohols, such as xylitol and sorbitol.

A disintegrant may be selected from, for example, alginic acid, carbon dioxide, carboxymethylcellulose calcium, carboxymethylcellulose sodium, microcrystalline cellulose, powdered cellulose, croscarmelose sodium, crospovidone, sodium docusate, gaur gum, hydroxypropyl cellulose, methylcellulose, polacrilin potassium, poloxamer, povidone, sodium alginate, sodium glycine carbonate, sodium lauryl sulfate, sodium starch glycolate, starch, pregelatinized starch, low-substituted hydroxypropyl cellulose.

A glidant may be selected from, for example, calcium silicate, powdered cellulose, starch, talc, colloidal silicon dioxide.

A lubricant may be selected from, for example, magnesium stearate, stearic acid, sodium stearyl fumarate, magnesium lauryl sulphate, talc, polyethylene glycol, and glyceryl behenate.

A suitable essential oil may be selected from Bergamot oil (extracted from Citrus aurantium L. subsp. bergamia Wright et Arn.); Ylang ylang oil (extracted from Cananga odorata Hook. f. and Thoms.); Jasmine essential oil (extracted from Jasminum officinale L.). In one embodiment, a mixture of essential oils comprises equal portions totaling about 0.01% to about 1% w/w, preferably about 0.1% w/w of the total composition. Other essential oils are possible.

A suitable sweetener may be selected from sugars such as sucrose, lactose and glucose; cyclamate and salts thereof; saccharin and salts thereof; and aspartame.

Flavoring agents may be incorporated in the antiviral composition may be chosen from synthetic flavors oils and flavoring aromatics, natural oils, plant extracts. Examples include cinnamon oil, oil of wintergreen, peppermint oil, clove oil, bay oil, anise oil, eucalyptus, thyme oil, cedar leaf oil, nutmeg oil, sage oil or almond oil. Examples of flavoring agents include, but are not limited to, almond, apple, banana, berry, bubblegum, caramel, citrus, cherry, chocolate, coconut, grape, green tea, honey, lemon, licorice, lime, mango, maple, mint, orange, peach, pineapple, raisin, strawberry, vanilla, watermelon and combinations thereof. Flavors may be present in an amount ranging from about 0.05 to about 3 percent by weight based upon the weight of the composition. In some embodiments, the flavoring agent may be selected from natural or synthetic flavors such as, for example, strawberry flavor, wild cherry flavor, green apple flavor, spearmint flavor and peppermint flavor.

Absorption enhancers for use in accordance with certain embodiments of the present disclosure include, for example, Gelucire 44/14; Gelucire 50/13; Tagat TO; Tween 80; isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate, cyclodextrins, citric acid, sodium citrate, triacetin, combinations thereof, and the like. In certain preferred embodiments, the absorption enhancer is triacetin. In certain embodiments where an absorption enhancer is included in the formulation, the absorption enhancer is included in an amount of from about 0.001% to about 10% by weight of the formulation, preferably in an amount of about 0.01% to about 5% by weight of the formulation.

Routes of Administration and Dosage Forms

Administration to a subject of the antiviral product compositions according to the present disclosure may be via any common route so long as the target tissue is available via that route. The antiviral compositions may conveniently be presented in dosage unit form and may be prepared by any of the methods well known in the art of pharmacy. All methods include the step of bringing the antiviral composition (or the individual components thereof) into association with an excipient or carrier. In general, the antiviral compositions are prepared by uniformly and intimately bringing the active components into association with a liquid carrier or a finely divided solid carrier or both, and then, if necessary, shaping the product into the desired dosage form. In the antiviral compositions, the active components are included in an amount sufficient to produce the desired pharmacologic effect.

Oral Dosage Forms

The antiviral compositions containing the active components may be provided, in general, in the form of discrete units such as hard or soft capsules, tablets, troches or lozenges, each containing a predetermined amount of the active components; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

Liquid dosage forms for oral administration include, but are not limited to, pharmaceutically acceptable emulsions, solutions, suspensions, syrups and elixirs. In addition to the antiviral compositions active components described herein, any liquid dosage forms may contain inert diluents commonly used in the art. For instance, liquid formulations can contain water, alcohol, polyethylene glycol ethers, or any other pharmaceutically acceptable solvents. Solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof may also be present in the inventive compositions. Additionally, oral compositions can include adjuvants such as wetting agents, emulsifying and suspending agents, sweetening, flavoring, and perfuming agents. When formulated as a suspension, the inventive compositions contain the cannabinoid extract and suspending agents, for example, ethoxylated isostearyl alcohols, polyoxyethylene sorbitol, sorbitan esters, microcrystalline cellulose, aluminum metahydroxide, bentonite, agar-agar, tragacanth, and mixtures thereof.

Aqueous suspensions normally contain the antiviral composition active components in admixture with excipients suitable for the manufacture of aqueous suspensions. Such excipients may be (a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia; (b) dispersing or wetting agents which may be (b.1) a naturally-occurring phosphatide such as lecithin, (b.2) a condensation product of an alkylene oxide with a fatty acid, for example, polyoxyethylene stearate, (b.3) a condensation product of ethylene oxide with a long chain aliphatic alcohol, for example heptadecaethyleneoxycetanol, (b.4) a condensation product of ethylene oxide with a partial ester derived from a fatty acid and a hexitol such as polyoxyethylene sorbitol monooleate, or (b.5) a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the antiviral composition active components in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the antiviral composition active components separately in admixture with a dispersing or wetting agent, a suspending agent and one or more preservatives. Suitable dispersing or wetting agents and suspending agents are exemplified by those already described herein. Additional excipients, for example, those sweetening, flavoring and coloring agents described above may also be present, including each of those described herein.

The pharmaceutical compositions of the disclosure may also be in the form of oil-in-water emulsions. The oily phase may be a vegetable oil such as olive oil or arachis oils, or a mineral oil such as liquid paraffin or a mixture thereof.

Suitable emulsifying agents may be (a) naturally-occurring gums such as gum acacia and gum tragacanth, (b) naturally-occurring phosphatides such as soybean and lecithin, (c) esters or partial esters derived from fatty acids and hexitol anhydrides, for example, sorbitan monooleate, (d) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a preservative and flavoring and coloring agents.

Solid dosage forms suitable for oral administration include capsules, tablets, pills, powders, and granules. The antiviral compositions disclosed herein may also be formulated into candies, lollipops, lozenges, etc. In such solid dosage forms, the antiviral compositions may be mixed with at least one pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For capsules, tablets and pills, the dosage form can also comprise buffering agents.

In some embodiments, antiviral compositions for oral use may be in the form of hardgelatin or HPMC capsules wherein the active components are mixed with an inert solid diluent, for example pregelatinized starch, calcium carbonate, calcium phosphate or kaolin, or dispensed via a pellet formulation. They may also be in the form of soft gelatin capsules wherein the active ingredient is mixed with water or an oil medium, for example peanut oil, liquid paraffin, medium chain triglycerides or olive oil.

The tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

Intranasal Dosage Forms

An intranasal formulation may include the antiviral composition and one or more excipients or additives. In some embodiments, the intranasal formulation further comprises a pharmaceutically acceptable liquid carrier to dissolve or suspend the ingredients. Exemplary pharmaceutically acceptable liquid carriers include purified water; and pharmaceutically acceptable organic liquid carriers, for example glycerin; propylene glycol; a lower molecular weight polyethylene glycol (e.g., polyethylene glycol 200, polyethylene glycol 300, polyethylene glycol 400, polyethylene glycol 540, polyethylene glycol 600, and the like); ethanol; propylene carbonate; or a combination thereof.

The intranasal formulation can optionally further comprise an intranasal formulation excipient such as a buffering agent, a flavoring agent, a sweetening agent, a tonicity agent, an antimicrobial preservative, an antimicrobial preservative synergist, a surfactant, an emulsifier, a solubilizer, an absorption enhancer, or a combination thereof. In some instances, a single compound or material will meet two or more of the foregoing general classifications. For example, a compound may function as both an emulsifier and a surfactant.

The intranasal dosage formulation can further include an antimicrobial preservative to prevent the unwanted growth of bacteria, molds, fungi, or yeast. Examples of suitable antimicrobial preservatives include benzyl alcohol, benzalkonium chloride, benzoic acid alkali metal salts (e.g., sodium benzoate), sorbic acid alkali metal salts (e.g., potassium sorbate), sodium erythorbate, sodium nitrite, calcium sorbate, butylated hydroxyanisole (BHA), butylated hydroxytoluene (BHT), parabens (e.g., lower alkyl esters of para-hydroxybenzoic acid), alkali metal salts of parabens including sodium and potassium salts of methyl-, ethyl-, propyl-, or butylparaben, or a combination thereof. Specific antimicrobial preservatives include benzyl alcohol, benzalkonium chloride, or a combination thereof.

Dosing and Dosing Schedules

One of ordinary skill will appreciate that effective amounts of the components in the formulations used in the methods of the present disclosure can be determined empirically. It will be understood that, when administered to a patient, the total daily usage of the formulation of the present disclosure will be decided by the attending physician or other medical professional within the scope of sound medical judgment. The specific therapeutically effective dose level for any patient will depend upon a variety of factors: the type and degree of the response to be achieved; the activity of the specific composition employed; the age, body weight, general health, sex and diet of the patient; the duration of the treatment; drugs used in combination or coincidental with the method of the present disclosure; and like factors well known in the medical arts.

In some embodiments, an amount of spikenard provided per dose ranges from between about 150 mg to about 600 mg. In other embodiments, an amount of spikenard provided per dose ranges from between about 200 mg to about 500 mg. In yet other embodiments, an amount of spikenard provided per dose ranges from between about 250 mg to about 350 mg. In further embodiments, an amount of spikenard provided per dose is about 350 mg. In some embodiments spikenard is administered in an amount ranging from about 4.5 mg/kg/day to about 24 mg/kg/day. In some embodiments, spikenard is administered in an amount ranging from about 6 mg/kg/day to about 15 mg/kg/day.

In some embodiments, an amount of cannabidiol provided per dose ranges from between about 100 mg to about 400 mg. In other embodiments, an amount of cannabidiol provided per dose ranges from between about 150 mg to about 250 mg. In yet other embodiments, an amount of cannabidiol provided per dose ranges from between about 175 mg to about 225 mg. In further embodiments, an amount of cannabidiol provided per dose is about 200 mg. In some embodiments cannabidiol is administered in an amount ranging from about 3 mg/kg/day to about 16 mg/kg/day. In some embodiments, cannabidiol is administered in an amount ranging from about 4 mg/kg/day to about 12 mg/kg/day.

In some embodiments, the antiviral composition is administered once per day. In other embodiments, the antiviral composition is administered twice per day. In other embodiments, the antiviral composition is administered at least three times per day. In some embodiments, the antiviral composition may be administered every 12 hours. In other embodiments, the antiviral composition may be administered every 8 hours. In yet other embodiments, the antiviral composition may be administered every 4 hours. In even further embodiments, the antiviral composition may be administered on an as-needed basis, but where the number of dosages in a 24-hour period does not exceed a predetermined number of doses or a predetermined amount of each active component. In some embodiments, the antiviral composition is administered with food. In other embodiments, the antiviral composition is administered while in a fasted state.

In case of nasal administration, the antiviral composition according to the present disclosure can be administered according to the specific, individual circumstances and the requirements of the patient in need thereof. In one embodiment, the antiviral composition is administered by nasal administration to one nostril of the nose but not to the other nostril, either once daily or more once daily, for example twice or thrice daily. In another embodiment, the antiviral composition is administered by nasal administration consecutively to both nostrils of the nose either once daily or more than once daily, for example twice or thrice daily. In case of such consecutive administration to both nostrils of the nose, one or more sprays of the antiviral composition is administered to one nostril and consecutively, one or more sprays of the pharmaceutical formulation is administered to the other nostril. In a further embodiment, one spray of the antiviral composition is administered to both nostrils once daily. In a further embodiment, one spray of the antiviral composition is administered to both nostrils twice daily, for example one spray to each nostril in the morning and one spray to each nostril in the evening. In a further embodiment two sprays of the antiviral composition is administered to both nostrils once daily, for example two sprays to each nostril either in the morning or in the evening. In a further embodiment, two sprays of the antiviral composition is administered to both nostrils twice daily, for example two sprays to each nostril in the morning and two sprays to each nostril in the evening.

Any of the antiviral compositions described herein can be provided in a unit dosage form. A unit dosage is a total amount of all of the active components within the antiviral compositions, which may delivered alone or in combination with other components, and which is to be administered to a subject at or about one time point. Other components which can be included with a unit dosage include, but are not limited, food carriers (e.g. gelatin or gelatin substitutes), dairy products, oils, beverages, such as denoted herein. A unit dosage of an antiviral composition may comprise about 1000, 1250, 1500, 1750, 2000, 2500, 3000, 3250, 3500, 3750, 4000, 4250, 4500, 4750, 5000, 5250, 5500, 5750 or more milligrams (mg) of combined active components. A unit dosage of an antiviral composition may comprise at least about 1000, 1250, 1500, 1750, 2000, 2500, 3000, 3250, 3500, 3750, 4000, 4250, 4500, 4750, 5000, 5250, 5500, 5750 or more milligrams (mg) of combined active components. A unit dosage of a antiviral composition may comprise at most about 1000, 1250, 1500, 1750, 2000, 2500, 3000, 3250, 3500, 3750, 4000, 4250, 4500, 4750, 5000, 5250, 5500, 5750 or more milligrams (mg) of combined active components. A unit dosage can be an hourly dosage. A unit dosage can be a daily dosage. A unit dosage can provide about 1/24, 1/12, ⅛, ⅙, ¼, ⅓, ½, or all of a daily dosage of one or more antiviral compositions for a subject in need thereof. As noted herein, a unit dosage can take the form of a tablet, gel, liquid, food product, food bar, container of liquid of defined volume, or other forms described herein, packaged for one-time consumption or administration.

Methods of Treatment

According to the methods of the disclosure, the antiviral compositions may be administrated to treat, prevent, or reduce the symptoms of CoV. In some embodiments, the antiviral composition according to present disclosure provides clinical improvement, where the clinical improvement is characterized by any one or more of the below parameters: (i) reduction in duration of supplemental oxygen requirement; (ii) reduction in duration of ECMO or mechanical ventilation; (iii) reduction in time to alleviation of cough; (iv) reduction in time to normalization of fever without use of antipyretics; (v) reduction in duration of hospitalization; or (vi) reduction in time to first negative SARS-CoV-2 RT-PCR in upper or lower respiratory tract specimen.

In some embodiments, the administration of the antiviral composition of the present disclosure to a subject in need of treatment thereof (e.g., to a patient infected with SARS-CoV-2 virus infection) provides clinical improvement in signs and symptoms, wherein the clinical improvement is characterized by a patient meeting at least one point, preferably at least two point improvement in disease severity rating on an ordinal scale after administration of the composition to the subject, the said ordinal scale defined as (i) not hospitalized with resumption of normal activities; (ii) not hospitalized, but unable to resume normal activities; (iii) hospitalized, not requiring supplemental oxygen; (iv) hospitalized, requiring supplemental oxygen; (v) hospitalized, requiring nasal high-flow oxygen therapy or noninvasive mechanical ventilation, or both; or (vi) hospitalized, requiring ECMO or invasive mechanical ventilation, or both.

In some embodiments, the administration of the antiviral composition of the present disclosure to a subject in need of treatment thereof (e.g., to a patient infected with SARS-CoV-2 virus infection) provides clinical improvement in signs and symptoms of SARS-CoV-2 virus infection in a patient. In another embodiment the extract or pharmaceutical composition according to present disclosure provides clinical improvement characterized by radiological improvement with a documented virological clearance in 2 samples tested at least 24 hours apart. In another embodiment the extract or pharmaceutical composition according to present disclosure provides clinical improvement characterized by time to normalization of fever without use of one or more antipyretics in last 24 hours. In another embodiment the extract or pharmaceutical composition according to present disclosure provides clinical improvement is further characterized by a negative SARS-CoV-2 RT-PCR of an upper or lower respiratory tract specimen.

In some embodiments, the antiviral compositions disclosed herein may be administered in a combination therapy, i.e., either simultaneously in single or separate dosage forms or in separate dosage forms within hours or days of each other. Examples of compounds/drugs used in such combination therapies include a nucleoside analogue, a reverse transcriptase inhibitor, a protease inhibitor, and a neuraminidase inhibitor. Suitable examples of chemical antiviral agents for incorporation in the conjugates of the invention include e.g. the nucleotide reverse transcriptase inhibitor (NtRTI) tenofovir disoproxil fumarate; the non-nucleoside reverse transcriptase inhibitors (NNRTIs) nevirapine, delavirdine and efavirenz; the protease inhibitors saquinavir, ritonavir, indinavir, nelfinavir, amprenavir, lopinavir, darunavir and atazanavir; the neuraminidase inhibitors peramivir, zanamivir (Tamiflu) and oseltamivir (Relenza); amantadine and rimantadine; and adefovir dipivoxil, famciclovir, penciclovir, imiquimod, docosanole, foscarnet (PFA), maribavir, BAY 38-4766, GW275175X, MVE-1, MVE-2, AM-3, AM-5, mannozym, bropirimine, 3,6-bis(2-p-peridinoethoxy) acridine trihydrochloride, phenyleneamine, 2-amino-5-halo-6-aryl-4(3H)-pyrimidinones, 2-amino-5-bromo-6-methyl-4(3H)-pyrimidinone, 7,8-didehydro-7-methyl-8-thioxoguanosine, 7-deazaguanosine, melatonin, 8-chloro-7-deazaguanosine, CL246,738, glycyrrhizin, pleconaril, bananin, iodobananin, vanillinbananin, ansabananin, eubananin, adeninobananin, cloroquine, valinomycin, and compounds as detailed in WO2006119646, WO2005107742, EP1736478, EP1707571, WO2004062676, EP1674104, WO2006060774, WO2006121767, the disclosures of which are hereby incorporated by reference herein in their entireties.

Suitable antiviral nucleoside analogues for incorporation in the conjugates of the invention include nucleoside analogues having an altered sugar, base or both. Examples of suitable nucleoside analogues include e.g. idoxuridine, aciclovir (acyclovir or acycloguansoine), valaciclovir (valacyclovir), ganciclovir, valganciclovir, adenosine arabino side (AraA, Vidarabine), AraA monophosphate, cytosine arabinoside (AraC, cytarabine), cytosine arabinoside monophosphate (Ara-CMP), azidothymidine (AZT), 1-beta-D-ribofuranosyl-1,2,4-triazole-3-carboxamide (ribavirin or RBV), 5-ethynyl-1-beta-D-ribofuranosylimidazole-4-carboxamide (EICAR), EICAR-monophosphate, ribamidine, ribavirin 2′,3′,5′-acetate, ribavirin-5′-sulfamate, ribavirin 5′-triphosphate, ribavirin 5′-monophosphate, ZX-2401, mycophenolic acid, tiazofurin, tiazofurin-5′-monophosphate, tiazofurin 2′,3′,5′-acetate, 7-thia-8-oxoguanosine, selenazofurin, pyrazofurin, furanonaphthoquinone derivatives, merimepodib (VX497), viramidine, 6-azauridine, 9-(2-phosphonylmethoxyethyl)guanine (PMEG), (S)-9-(3-hydroxy-2-phosphonylmethoxypropyl)adenine (HPMPA), 9-(2-phosphonylmethoxyethyl)adenine (PMEA), 9-(2-phosphonylmethoxyethyl)-2,6-diaminopurine (PMEDAP), didenosine (DDI), dideoxycytosine (DDC), stavudine (d4T), Epivir (3TC), abacavir (ABC), iodo-deozyuridine (DU), and bromovinyl deoxiuridine (BVDU or brivudin), (S)-1-(3-hydroxy phosphonylmethoxypropyl)cytosine (HPMPC, cidofovir, CDV or Vistide®), cyclic HPMPC, hexadecyloxypropyl-cidofovir (HDP-CDV, or CMX001), 3-deazaguanine (3-DG), 3-deazauridine, 9-(S)-(2,3-dihydroxypropyl)adenine ((S)-DHPA), zidovudine, didanosine, zalcitabine, stavudine, lamivudine, abacavir and emtricitabine.

### Examples

| Component | Amount by weight (%) |
|---|---|
| oregano oil | about 1-about 20% |
| Artemisinin | about 1-about 20% |
| spikenard | about 1-about 20% |
| salvia sclarea | about 1-about 20% |
| zinc | about 1-about 20% |
| copper | about 1-about 20% |
| vitamin D | about 1-about 20% |
| vitamin C | about 1-about 20% |
| cardamom oil | about 10-about 30% |

| Component | Amount by weight (%) |
|---|---|
| oregano oil | about 5-about 15% |
| Artemisinin | about 5-about 15% |
| spikenard | about 5-about 15% |
| salvia sclarea | about 5-about 15% |
| zinc | about 5-about 15% |
| copper | about 5-about 15% |
| vitamin D | about 5-about 15% |
| vitamin C | about 5-about 15% |
| cardamom oil | about 10-about 30% |

| Component | Amount by weight (%) |
|---|---|
| oregano oil | about 8-about 12% |
| Artemisinin | about 8-about 12% |
| spikenard | about 8-about 12% |
| salvia sclarea | about 8-about 12% |
| zinc | about 8-about 12% |
| copper | about 8-about 12% |
| vitamin D | about 8-about 12% |
| vitamin C | about 8-about 12% |
| cardamom oil | about 10-about 30% |

| Component | Amount by weight (%) |
|---|---|
| oregano oil | about 10% |
| Artemisinin | about 10% |
| spikenard | about 10% |
| salvia sclarea | about 10% |
| zinc | about 10% |
| copper | about 10% |
| vitamin D | about 10% |
| vitamin C | about 10% |
| cardamom oil | about 20% |

| Component | Amount by weight (%) |
|---|---|
| oregano oil | about 1-about 20% |
| Artemisinin | about 1-about 20% |
| spikenard | about 1-about 20% |
| salvia sclarea | about 1-about 20% |
| zinc | about 1-about 20% |
| copper | about 1-about 20% |
| vitamin D | about 1-about 20% |
| vitamin C | about 1-about 20% |
| cardamom oil | about 10-about 30% |
| cannabidiol | about 5-about 15% |

| Component | Amount by weight (%) |
|---|---|
| oregano oil | about 5-about 15% |
| Artemisinin | about 5-about 15% |
| spikenard | about 5-about 15% |
| salvia sclarea | about 5-about 15% |
| zinc | about 5-about 15% |
| copper | about 5-about 15% |
| vitamin D | about 5-about 15% |
| vitamin C | about 5-about 15% |
| cardamom oil | about 10-about 30% |
| cannabidiol | about 5-about 15% |

| Component | Amount by weight (%) |
|---|---|
| oregano oil | about 10% |
| Artemisinin | about 10% |
| spikenard | about 10% |
| salvia sclarea | about 10% |
| zinc | about 10% |
| copper | about 10% |
| vitamin D | about 10% |
| vitamin C | about 10% |
| cardamom oil | about 10% |
| cannabidiol | about 10% |

1. A antiviral composition comprising: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

2. The antiviral composition of claim 1, wherein the at least one mineral is selected from zinc or copper.

3. The antiviral composition of claim 1, wherein the antiviral composition comprises a therapeutically effective amount of at least two minerals.

4. The antiviral composition of claim 3, wherein the at least two minerals are zinc and copper.

5. The antiviral composition of claim 1, wherein the at least one vitamin is selected from Vitamin D or Vitamin C.

6. The antiviral composition of claim 1, wherein the antiviral composition comprises a therapeutically effective amount of at least two vitamins.

7. The antiviral composition of claim 6, wherein the at least two vitamins are Vitamin D and Vitamin C.

8. The antiviral composition of claim 1, wherein the one or more Artemisinins are selected from the group consisting of dihydroartemisinin (DHA), artemether, artesunate, artemisone, arteether, and artelinic acid.

9. The antiviral composition of claim 1, wherein the antiviral composition further comprises a cannabidiol.

10. The antiviral composition of claim 1, further comprising a pharmaceutically acceptable carrier or excipient.

11. A method of treating a CoV infection comprising administering the antiviral composition of claim 1 to a subject in need of treatment thereof.

12. A method of reducing a viral load comprising administering the antiviral composition of claim 1 to a subject in need of treatment thereof.

13. A method of reducing a symptom of a CoV infection comprising administering the antiviral composition of claim 1 to a subject in need of treatment thereof.

14. A method of preventing a CoV infection comprising administering the antiviral composition of claim 1 to a subject in need of treatment thereof.

15. A method of preventing hospitalization of a patient infected with CoV comprising administering the antiviral composition of claim 1 to a subject in need of treatment thereof.

16. A antiviral composition consisting essentially of: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof.

17. A method of treating a CoV infection comprising administering the antiviral composition of claim 16 to a subject in need of treatment thereof.

18. A method of reducing a viral load comprising administering the antiviral composition of claim 16 to a subject in need of treatment thereof.

19. A method of reducing a symptom of a CoV infection comprising administering the antiviral composition of claim 16 to a subject in need of treatment thereof.

20. An intranasal dosage form comprising (i) at least 5 components selected from: a therapeutically effect amount of spikenard or an extract thereof; a therapeutically effective amount of at least one mineral; a therapeutically effective amount of at least one vitamin; a therapeutically effective amount of oregano oil or at least one active component included within oregano oil; a therapeutically effective amount of one or more Artemisinins or derivatives or analogs thereof; a therapeutically effective amount of one or more Salvia species or an extract thereof; and a therapeutically effective amount of cardamom oil or an extract thereof; and (ii) a pharmaceutically acceptable carrier or excipient.
